(19) United States
(12) Patent Application Publication
(10) Pub. No.: US 2025/0352126 A1
(43) Pub. Date: Nov. 20, 2025

(54) IDENTIFYING RISK LEVEL FOR SEIZURE ACTIVITY BASED ON SLEEP STATES

(71) Applicant: NEUROVIGIL, INC., Moffett Field, CA (US)

(72) Inventor: Philip Low, Moffett Field, CA (US)

(73) Assignee: NEUROVIGIL, INC., Moffett Field, CA (US)

(21) Appl. No.: 19/291,464

(22) Filed: Aug. 5, 2025

Related U.S. Application Data
(63) Continuation of application No. PCT/US2024/015563, filed on Feb. 13, 2024.
(60) Provisional application No. 63/484,570, filed on Feb. 13, 2023.

(51) Int. Cl. A61B 5/00; A61B 5/291

(52) U.S. Cl. CPC A61B 5/4094; A61B 5/291; A61B 5/4812; A61B 5/7275; A61B 5/742

(57) ABSTRACT

A computer-implemented method can include receiving data indicative of brainwave activity over a particular time period. The method can also include determining, based on the data, at least one metric associated with at least one sleep state for a subject. Additionally, the method can include determining, based on the at least one metric, a risk level associated with seizure activity for the subject. The method can further include generating an output indicating the risk level.

### CROSS-REFERENCES TO RELATED APPLICATIONS

The present application is a continuation of International Patent Application No. PCT/US2024/015563, filed on Feb. 13, 2024, which claims the benefit of U.S. Provisional Application No. 63/484,570, filed on Feb. 13, 2023, and entitled “Identifying Risk Level for Seizure Activity Based on Sleep States”. The entire disclosures of the aforementioned applications are incorporated by reference herein in their entireties for all purposes.

### TECHNICAL FIELD

The present disclosure relates generally to analyzing physiological data and, more particularly (although not necessarily exclusively), to identifying risk levels for seizure activity based on sleep states.

### BACKGROUND

An electroencephalogram (EEG) is a tool used to measure electrical activity produced by the brain. The functional activity of the brain is collected by electrodes placed on the scalp of a subject. Conventional monitoring and diagnostic equipment include several electrodes mounted on the subject, which tap the brain signals and transmit the signals via cables to amplifier units. The EEG signals obtained can be used to diagnose and monitor various conditions that affect the brain, such as epilepsy.

Epilepsy is a brain disorder that causes seizures. There are many types of epilepsy, which can be diagnosed based on types of seizures suffered by a subject. Examples of the types of epilepsy can include focal epilepsy, generalized epilepsy, and unknown epilepsy. The subject can experience more than one type of seizure, and seizures may vary in manifestation by, for example, occurring with varying intensity, duration, symptoms, etc. Treatments for epilepsy can include medication, but subjects can often be resistant to the medication. Further, the medication may increase the likelihood of sleep deprivation or substance abuse, which may increase the likelihood of seizures. Thus, epilepsy can be a complex disorder that can be difficult to diagnose and treat.

It can further be difficult to predict when people with seizures will experience seizures. For some subjects, subtle changes in heart rate, skin electrical conduction, or breathing patterns, can be indicators used to predict seizure activity immediately before the onset of a seizure. Additionally, conventional systems have applied machine learning techniques to EEG data to extract features that can be used for seizure prediction. However, the complexity of seizure activity can make it difficult to extract features that can be used to accurately predict seizures for populations of subjects (e.g., especially for subjects outside a population used in training the machine learning techniques). Therefore, there is a need for a holistic and accurate approach to seizure prediction. Additionally, there is a further need for the prediction to occur earlier than immediately before onset of the seizure to enable preparation, intervention, or treatment.

### BRIEF SUMMARY

Aspects of the present disclosure relate to identifying risk level for seizure activity based on sleep states. One aspect relates to a computer-implemented method. The method includes receiving data indicative of brainwave activity over a particular time period, determining, based on the data, at least one metric associated with at least one sleep state for a subject, determining, based on the at least one metric, a risk level associated with seizure activity for the subject, and generating an output indicating the risk level.

In some embodiments, the at least one metric is an amount of time for a particular sleep state. In some embodiments, the particular sleep state is a rapid eye movement (REM) state of sleep.

In some embodiments, the data is received from a RF transmitter-receiver associated with a multi-electrode device. In some embodiments, the particular time period is a first time period. In some embodiments, the risk level is a prediction of a likelihood of the subject experiencing the seizure activity for a second time period. In some embodiments, the second time period occurs subsequent to the first time period.

In some embodiments, the output is a first output. In some embodiments, the method further includes identifying, based on the risk level, a treatment recommendation, and generating a second output indicating the treatment recommendation, the treatment recommendation usable to reduce the risk level.

In some embodiments, the at least one metric is a first metric. In some embodiments, determining, based on the at least one metric, the risk level further includes determining a second metric for a healthy population based on historical data, identifying a statistically significant difference between the first metric and the second metric based on a comparison of the first metric and the second metric, and determining, based on the statistically significant difference, the risk level.

In some embodiments, generating the output indicating the risk level further includes providing the output indicating a high-risk level in a first color, providing the output indicating a moderate risk level in a second color, and providing the output indicating a low risk level in a third color.

One aspect relates to a system. The system includes one or more data processors and a non-transitory computer readable storage medium. The computer readable storage medium contains instructions which, when executed on the one or more data processors, cause the one or more data processors to receive data indicative of brainwave activity over a particular time period, determine, based on the data, at least one metric associated with at least one sleep state for a subject, determine, based on the at least one metric, a risk level associated with seizure activity for the subject, and generate an output indicating the risk level.

In some embodiments, the at least one metric is an amount of time for a particular sleep state. In some embodiments, the particular sleep state is a rapid eye movement (REM) state of sleep.

In some embodiments, the data is received from a RF transmitter-receiver associated with a multi-electrode device. In some embodiments, the particular time period is a first time period. In some embodiments, the risk level is a prediction of a likelihood of the subject experiencing the seizure activity for a second time period. In some embodiments, the second time period occurs subsequent to the first time period.

In some embodiments, the output is a first output. In some embodiments, the instructions, when executed on the one or more data processors, cause the one or more data processors to identify, based on the risk level, a treatment recommendation, and generate a second output indicating the treatment recommendation, the treatment recommendation usable to reduce the risk level.

In some embodiments, the at least one metric is a first metric. In some embodiments, determining, based on the at least one metric, the risk level further includes determining a second metric for a healthy population based on historical data, identifying a statistically significant difference between the first metric and the second metric based on a comparison of the first metric and the second metric, and determining, based on the statistically significant difference, the risk level.

In some embodiments, generating the output indicating the risk level further includes providing the output indicating a high-risk level in a first color, providing the output indicating a moderate risk level in a second color, and providing the output indicating a low risk level in a third color.

One aspect relates to a computer-program product tangibly embodied in a non-transitory machine-readable storage medium. The computer-program product comprises instructions that cause one or more data processors to receive data indicative of brainwave activity over a particular time period, determine, based on the data, at least one metric associated with at least one sleep state for a subject, determine, based on the at least one metric, a risk level associated with seizure activity for the subject, and generate an output indicating the risk level.

In some embodiments, the at least one metric is an amount of time for a particular sleep state. In some embodiments, the particular sleep state is a rapid eye movement (REM) state of sleep.

In some embodiments, the data is received from a RF transmitter-receiver associated with a multi-electrode device. In some embodiments, the particular time period is a first time period. In some embodiments, the risk level is a prediction of a likelihood of the subject experiencing the seizure activity for a second time period. In some embodiments, the second time period occurs subsequent to the first time period.

In some embodiments, the output is a first output. In some embodiments, the instructions cause the instructions further cause one or more data processors to identify, based on the risk level, a treatment recommendation, and generate a second output indicating the treatment recommendation, the treatment recommendation usable to reduce the risk level.

In some embodiments, the at least one metric is a first metric. In some embodiments, determining, based on the at least one metric, the risk level further includes determining a second metric for a healthy population based on historical data, identifying a statistically significant difference between the first metric and the second metric based on a comparison of the first metric and the second metric, and determining, based on the statistically significant difference, the risk level.

In some embodiments, generating the output indicating the risk level further includes providing the output indicating a high-risk level in a first color, providing the output indicating a moderate risk level in a second color, and providing the output indicating a low risk level in a third color.

### BRIEF DESCRIPTION OF THE DRAWINGS

FIG. 1 is a block diagram of an example of a system for acquiring physiological data according to one example of the present disclosure.

FIG. 2 is a block diagram of an example of a system for identifying risk level for seizure activity based on sleep states according to one example of the present disclosure.

FIG. 3 is a block diagram of an example of a computing system for identifying risk level for seizure activity based on sleep states according to one example of the present disclosure.

FIG. 4 is a flowchart of a process for identifying risk level for seizure activity based on sleep states according to one example of the present disclosure.

### DETAILED DESCRIPTION

Certain aspects and examples of the present disclosure relate to a system and method for predicting a risk level for seizure activity within a particular timeframe based on analysis of physiological data. The risk level can be a likelihood that a subject will exhibit seizure activity (e.g., within a predefined time period). The seizure activity may include neural activity (e.g., as collected via one or more electroencephalogram electrodes) that are consistent with a seizure and/or a clinical episode consistent with a seizure. Various environmental, physiological, or other suitable factors can be used to determine the risk level. Physiological data used to predict risk level can include electroencephalography (EEG) data, electrocardiography (ECG) data, electromyography (EMG) data, electrooculography (EOG) data, or other suitable physiological data. The physiological data can be obtained via a physiological data acquisition assembly that includes at least a single channel of physiological data with at least one reference electrode and at least one active electrode in close proximity. The assembly can be worn by a subject. For example, the assembly can include a patch configurable to be positioned on (e.g., adhered to) a user's forehead. Additionally, the patch can have an adhesive film to which the electrodes can be attached to collect physiological data.

In one example of the present disclosure, a risk level for seizure activity can be identified based on EEG data indicative of sleep states. The sleep states can be any distinguishable sleep or wakefulness that are representative of behavioral, physical, or signal characteristics. In some instances, EEG data is processed to infer—for each of multiple time intervals—a category that indicates a prediction as to whether the subject is awake or asleep, and potentially—if the subject is estimated as being asleep—a particular type or stage of sleep. The inference can be made based on—for each of the multiple time intervals—transforming time-domain electrical signals into frequency-domain intensity or power value. Features may be defined as cumulative or maximum intensity or power values within various frequency bands. Sleep states may then be inferred based on absolute or relative values of one or more features.

For example, a wake sleep state can be detected or defined by processing EEG data to detect signals within one or more particular frequency bands (e.g., a band that extends between about thirteen and about sixty hertz (Hz) and amplitudes of at least about thirty microvolts (μV) (i.e., Beta waves). The frequencies and amplitudes can be determined by transforming the time-domain electrical signals to the frequency-domain via mathematical transformations (e.g., Fourier Transform) or other suitable techniques. In some examples, additional sleep states can be characterized by stage one, stage two, stage three, and rapid eye movement (REM). A frequency band for detecting stage one sleep from EEG data can be defined to correspond to a particular type of wave and/or sleep stage. For example, a frequency band may be defined to extend between three to eight Hz. Detection algorithms may be configured in the time or frequency domain to detect signatures that support predictions as to whether a subject is in a given stage of sleep (e.g., stage one sleep). To illustrate, if amplitudes in the three to eight Hz band are between fifty to one-hundred μV (i.e., Theta waves), it may be inferred that the subject was in stage one sleep. Additional characteristics of sleep states, such as sleep spindles and K-complexes, can be discerned via the detection algorithms to predict sleep states. For example, a high frequency band (e.g., a frequency band of around fifteen Hz) that, in the time domain, lasts for less than two seconds may be detected as a sleep spindle. Similarly, a low frequency band (e.g., a frequency band that extends between one and four Hz and amplitudes between one-hundred and two-hundred μV) (i.e., Delta waves) that, in the time-domain, lasts for about one second can be detected as a K-Complex. Therefore, if one or more portions of EEG data are detected as sleep spindles and are followed by or otherwise detected near one or more portions of EEG data detected as K-Complexes, it may be inferred that the subject was in stage two sleep. In another example, frequency bands extending between one to four Hz can be detected for significantly longer than two seconds (e.g., for twenty minutes), and from this, it may be predicted that the subject was in stage three sleep. Stage three sleep may also be referred to as slow-wave or delta sleep. Moreover, for the frequency bands that extend between about thirteen and about sixty hertz (Hz) and for amplitudes of at least about thirty μV (i.e., Beta waves) it may be predicted that the subject was in REM sleep. However, Beta waves may also be detected during the wake sleep state. Therefore, additional physiological data, physical or biological indicators, or other suitable data can be obtained and identified within the detection algorithms to differentiate between REM sleep and the wake sleep state. For example, EMG data may be obtained and a detection algorithm may detect phasic events (e.g. rapid eye movements and twitches of the limbs) or tonic phenomena (e.g. loss of tone in antigravity muscles), both of which can be indicative of REM sleep. The detection of phasic events or tonic phenomena can be compared or combined with EEG data to distinguish the REM sleep state from the wake sleep state or another sleep state.

In some examples, the sleep states can be characterized by REM sleep and non-REM sleep. For example, the stage one sleep, stage two sleep, and stage three sleep can be combined to be the non-REM sleep. Thus, a detection algorithm may detect the non-REM sleep by inferring that EEG data outside of the thirteen to sixty hertz (Hz) range and with amplitudes above or below about thirty μV is non-REM sleep and detect REM sleep by inferring that data within the thirteen to sixty Hz range and amplitude of about thirty is REM sleep.

EEG signals have typically been examined in time in series increments called epochs. For example, when the EEG signal is used for analyzing sleep, sleep may be segmented into one or more epochs to use for analysis. The epochs can be segmented into different sections using a scanning window, where the scanning window defines different sections of the time series increment. Code can move (incrementally or via shifting) the scanning window via a sliding or shifting window, where sections of the sliding window have overlapping or non-overlapping time series sequences. An epoch can alternatively span an entire time series, for example. In some examples, each epoch can be classified to correspond to a predicted sleep state that is represented. In some instances, prior to the classification, the epoch is normalized or double normalized based on (for example) frequency information, amplitude information, power, intensity, or other suitable features of the EEG data that can be correlated with sleep states. U.S. patent application Ser. No. 11/431,425, filed on May 9, 2006, which is hereby incorporated by reference for all purposes, discloses exemplary techniques for normalizing biological data.

In some instances, a given epoch may be classified as REM (or alternatively non-REM) sleep based on whether the power (or normalized or double-normalized power) in a given frequency band (e.g., in an absolute sense or relative to the power in one or more other frequency bands) exceeds a threshold. The threshold may be an absolute threshold, a threshold that is defined based on data from a population of subjects that have been diagnosed with a given condition, a threshold that is defined based on data from a population of subjects experiencing a given condition, or a threshold that is defined based on empirical data associated with the subject.

Any same or similar technique may be used to predict a different type of sleep stage. For example, intensities within one or more bands of an epoch of a normalized or double normalized may be used to predict whether a subject is in Stage 1, 2, 3, or REM sleep, whether there are spindles are in the sleep data, whether there are k-complexes in the sleep data, etc.

Any group of epochs initially classified as one sleep state can be split into multiple sub-classified sleep states according to increasing levels of classification detail. For example, a group of epochs classified as non-REM can be further split into stage one, stage two, three, or a combination thereof.

In some embodiments, artificial-intelligence techniques can be used to predict that a subject has a given sleep disorder, to predict a severity of a sleep disorder, or to predict an efficacy of treating a given sleep disorder. An artificial-intelligence technique may include implanting signal processing (e.g., that may include applying one or more signal transformations) and using one or more models or rules to generate an epoch-specific, night-specific or subject-specific prediction. For example, EEG signals may be collected across a sleep time period (e.g., a night). The EEG signals may be separated into epochs that correspond to absolute or relative time increments through the time period (e.g., 1-minute, 5-minute, or 10-minute time intervals), and a spectrum can be generated for each epoch, such that a power or intensity for each of various frequency bands may be identified for each time increment. Alternatively, a spectrogram can be generated for the time period, where the spectrogram identifies power or intensity values for various frequency bands for each of multiple time increments (e.g., 1-minute, 5-minute, or 10-minute time increments) in the time period. A set of features may be defined such that a feature indicates or corresponds to: (1) a power, intensity, or other suitable attribute of the frequency band in the spectrogram or spectrum; and/or (2) an intensity of spectrum or spectrogram, a derivative (e.g., across time) of the spectrum, or a double derivative (e.g., across time, across frequency or across both time and frequency) of the spectrum or spectrogram. The feature may (for example) predict a likelihood of a particular sleep stage or state (e.g., REM or non-REM). In some instances, a feature is defined based on other epoch-specific features. For example, a feature specific to a subject and/or a day or night may indicate a percentage of time or percentage of sleep predicted to be in a REM state.

An artificial-intelligence rule can be defined to predict REM deprivation and/or seizure propensity based on the features. For example, a clustering technique, support vector machine (SVM) technique), principal components technique, independent components technique, logistic regression technique, etc. may be used to predict—for each time epoch—whether the subject is in REM sleep (versus non-REM sleep or awake). In some instances, for each epoch, a likelihood of the subject being in REM sleep is generated, which may then be compared against a predefined or learned threshold to predict whether the subject is or was in REM sleep.

A rule can be defined to predict—based on the REM sleep predictions—as to whether the subject is (or was) sleep deprived. For example, the rule may indicate that the subject is (or was) sleep deprived) if less than a threshold percentage (e.g., 10%, 15%, 20%, 25%, 30%, or 35%) of the epochs are predicted to be REM sleep. In another example, a rule may indicate that the subject was or is sleep deprived by identifying that a length of time for REM sleep as predicted by the detection algorithms for one or more epochs is less than a predefined threshold for healthy or normal sleep or for a learned threshold for the subject. An additional rule may indicate that, for subsequent sleep cycles (i.e., a series of sleep cycles for a night of sleep), if a length of time or percentage of REM sleep does not increase by at least a threshold amount (e.g., 1 minute, 5 minutes, etc. or 5%, 10%, etc.) then it can be inferred that the subject was or is sleep deprived.

To identify the risk level, metrics can be extracted from EEG data for the sleep states and can be correlated with an effect on seizure activity. For example, the metrics can be averages, variances, skewness, etc. of frequency bands, amplitudes, or other suitable features of the EEG data. In other examples, the metrics can be statistical values derived from an epoch or a set of epochs such as a percentage of sleep predicted to be REM sleep. The metrics may also be extracted from EMG data, EOG data, ECG data, or other suitable physiological data obtained during a period of sleep. For example, the metrics may include heart rate, oxygen level in blood, eye or leg movement, etc.

Additionally, in some examples, characteristics of the subject may further be used to correlate the metrics with the effect on seizure activity. For example, a metric can be a ratio of REM to non-REM sleep and the metric can be correlated with an effect on seizure activity based on an age of the subject. Additional characteristics of the subject may include a medication or a dosage of the medication prescribed to the subject, a sleep disorder previously diagnosed for the subject, gender of the subject, etc.

In a particular example, sleep deprivation can be detected based on a predefined rule and the metric extracted from the EEG data can be associated with the rule. For example, the predefined rule can include detecting sleep deprivation if a percentage of REM sleep associated with a set of epochs is less than the threshold percentage. Therefore, a metric can be the percentage of REM sleep. Then, a classification algorithm (e.g., K-nearest neighbors, decision tree, support vector machine, etc.) or another suitable algorithm may be implemented to predict the risk level based on the percentage of REM sleep. Additional metrics may be extracted from the set of epochs and provided for use in the classification algorithm. The additional metrics may be other suitable indicators of sleep deprivation (e.g., a length of time for which the subject was predicted to be asleep). The classification algorithm may also receive the characteristics of the subject (e.g., age). Then, the classification algorithm may indicate, based on the percentage of REM sleep, the additional metrics, and/or the characteristics of the subject, the risk level of seizure activity for the subject. For example, the classification algorithm may indicate the risk level by classifying the risk level as high, moderate, or low.

Moreover, a typical sleep cycle for a healthy subject detected and analyzed via EEG data can be determined to last around ninety minutes to one hundred and ten minutes. For the typical sleep cycle, epochs for each sleep state in chronological order may indicate a typical sleep cycle occurs as stage one, stage two, stage three, stage two, and then REM. Additionally, for EEG data collected over a typical night of sleep for a healthy subject, the EEG data may include four to five sleep cycles in which time periods associated with REM sleep can increase for each subsequent sleep cycle. Further, in the typical sleep cycle, about five percent of the EEG data obtained can be associated with stage one, about forty-five percent can be associated with stage two, about twenty-five percent can be associated with stage three, and about twenty-five percent can be associate with REM. Thus, about seventy-five percent of the EEG data for the typical sleep cycle can be associated with non-REM sleep and about twenty-five percent can be associated with REM sleep.

Therefore, because the abnormal sleep activity (e.g., sleep deprivation) is a common trigger for seizure activity and typical sleep activity can be well-defined, EEG data associated with sleep states can provide a comprehensive mechanism for predicting the risk level for the seizure activity. The sleep states can be predicted based on frequency bands, amplitudes, or other suitable features of the EEG data. Then, artificial intelligence techniques or rules may be implemented to predict sleep deprivation of other suitable abnormal sleep activity. Additionally, metrics can be derived from data associated with the predictions of sleep states and/or sleep deprivation and then classified (i.e., by a classification algorithm) to predict a risk level of seizure activity for the subject. Further, the abnormal sleep activity may not immediately trigger the seizure activity, and therefore the risk level for seizure activity can be predicted, based on the EEG data, for a certain timeframe subsequent to the abnormal sleep activity. Thus, examples of the present disclosure can provide an accurate and comprehensive method for predicting the risk level for seizure activity and can further predict the risk level for the certain timeframe to enable preparation for or prevention of the seizure activity.

Illustrative examples are given to introduce the reader to the general subject matter discussed herein and are not intended to limit the scope of the disclosed concepts. The following sections describe various additional features and examples with reference to the drawings in which like numerals indicate like elements, and directional descriptions are used to describe the illustrative aspects, but, like the illustrative aspects, should not be used to limit the present disclosure.

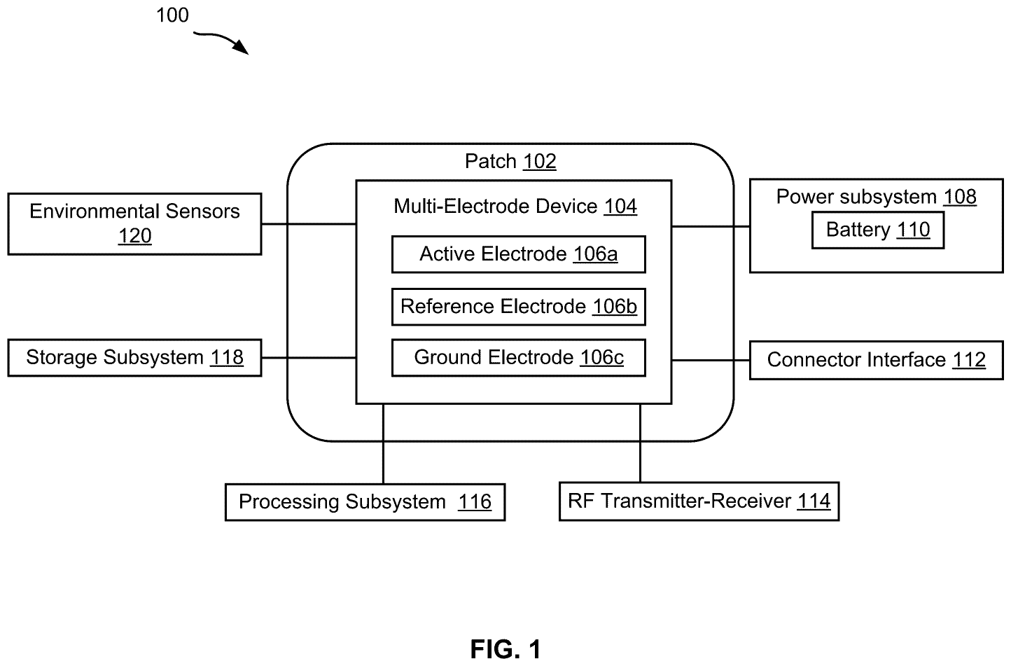

FIG. 1 is a block diagram of an example of a system for acquiring physiological data according to one example of the present disclosure. The system 100 can include a multi-electrode device 104, which can have one or more active electrodes 106a for collecting active signals and one or more reference electrodes 106b, which can collect respective reference signals. Additionally, the multi-electrode device 104 may include a ground electrode 106c. The electrodes 106a-c can be fixed in location within a device (e.g., patch 102) or movable (e.g., tethered to a device). The system 100 can further include a processing subsystem 116, a storage subsystem 118, a (radiofrequency) RF transmitter-receiver 114, a connector interface 112, a power subsystem 108, and environmental sensors 120, each of which can be communicatively coupled to or part of the multi-electrode device 104.

The processing subsystem 116 can be implemented as one or more integrated circuits, e.g., one or more single-core or multi-core microprocessors or microcontrollers, examples of which are known in the art. The processing subsystem 116 can control the operation of multi-electrode device 104 by executing a variety of programs in response to program code and may maintain multiple concurrently executing programs or processes. For example, the processing subsystem 116 may execute code that can control collection, analysis, application and/or transmission of physiological data (e.g. electroencephalogram (EEG) data, electromyography (EMG) data, etc.). Some or all of the program code can be stored in the processing subsystem 116 or the program code can be stored in storage media such as the storage subsystem 118. Additionally, the processing subsystem 116 may cause signals detected by the electrodes 106a-c of the multi-electrode device 104 to be amplified, filtered, or a combination thereof and may further store the signals along with recording details (e.g., a recording time or a user identifier). In some examples, the processing subsystem 116 can analyze the physiological data or signals to detect physiological correspondences. For example, the recorded signals can reveal frequency properties that correspond to sleep stages.

Additionally, the storage subsystem 118 can be implemented using, for example, magnetic storage media, flash memory, other semiconductor memory (e.g., DRAM, SRAM), or any other non-transitory storage medium, or a combination of media, and can include volatile and/or non-volatile media. In some examples, the storage subsystem 118 can store physiological data, information (e.g., identifying information or medical-history information) about a subject, or analysis variables (e.g., frequencies, amplitudes, etc.) obtained from the physiological data. The storage subsystem 118 can also store one or more programs that can be executed by the processing subsystem 116. The one or more programs may initiate or otherwise control collection, analysis, or transmission of the physiological data.

The RF transmitter-receiver 114 can enable the multi-electrode device 104 to communicate wirelessly with various interface devices, such as a phone, tablet, laptop, etc. The RF transmitter-receiver 114 can include a combination of hardware components including, for example, driver circuits, antennas, modulators, demodulators, encoders, decoders, other suitable analog and/or digital signal processing circuits and can also include software components. Various wireless communication protocols can be implemented via the RF transmitter-receiver 114 using the software components and associated hardware. RF transceiver components of the RF transmitter-receiver 114 can include an antenna and supporting circuitry to enable data communication over a wireless medium, such as Wi-Fi, Bluetooth®, or other suitable mediums for wireless data communication.

The connector interface 112 can enable the multi-electrode device 104 to communicate with various interface devices via a wired communication path, e.g., using Universal Serial Bus (USB), universal asynchronous receiver/transmitter (UART), or other protocols for wired data communication. In some examples, the connector interface 112 can provide a power port for allowing the multi-electrode device 104 to receive power. The connector interface 112 may also provide connections to transmit or receive the physiological data. For example, the physiological data can be transmitted to or from another device, such as another multi-electrode device, in analog or digital formats.

The environmental sensors 120 can include various electronic, mechanical, electromechanical, optical, or other devices that provide information related to external conditions around the multi-electrode device 104 or with respect to the subject. Any type and combination of the environmental sensors 120 can be used. For example, an accelerometer can be used to estimate whether a user is or is trying to sleep or otherwise estimate an activity state. In another example, an electrooculogram sensor can be used to detect eye-movement to assist in identifying a rapid eye movement (REM) sleep stage.

Additionally, the power subsystem 108 can provide power and power management capabilities for the multi-electrode device 104. For example, the power subsystem 108 can include a battery 110 and associated circuitry to distribute power from battery 440 to other components of the system 100 that may require electrical power.

It will be appreciated that system 100 is illustrative and that variations and modifications are possible. In an example, the processing subsystem 116 can execute code from the storage subsystem 118 for analyzing sleep states based on EEG data and predicting a risk level for seizure activity based on the analysis. Thus, the system 100 may further include a user interface to enable a user to directly interact with the device to, for example, receive the risk level. The risk level can be a likelihood of the subject experiencing seizure activity for a particular timeframe. The risk level may be displayed at the user interface in a color indicating whether the risk level is, for example, high, moderate, or low or the risk level may be displayed as a percentage or in another suitable format. Further, while the system 100 is described with reference to particular blocks, it is to be understood that these blocks are defined for convenience of description and are not intended to imply a particular physical arrangement of component parts.

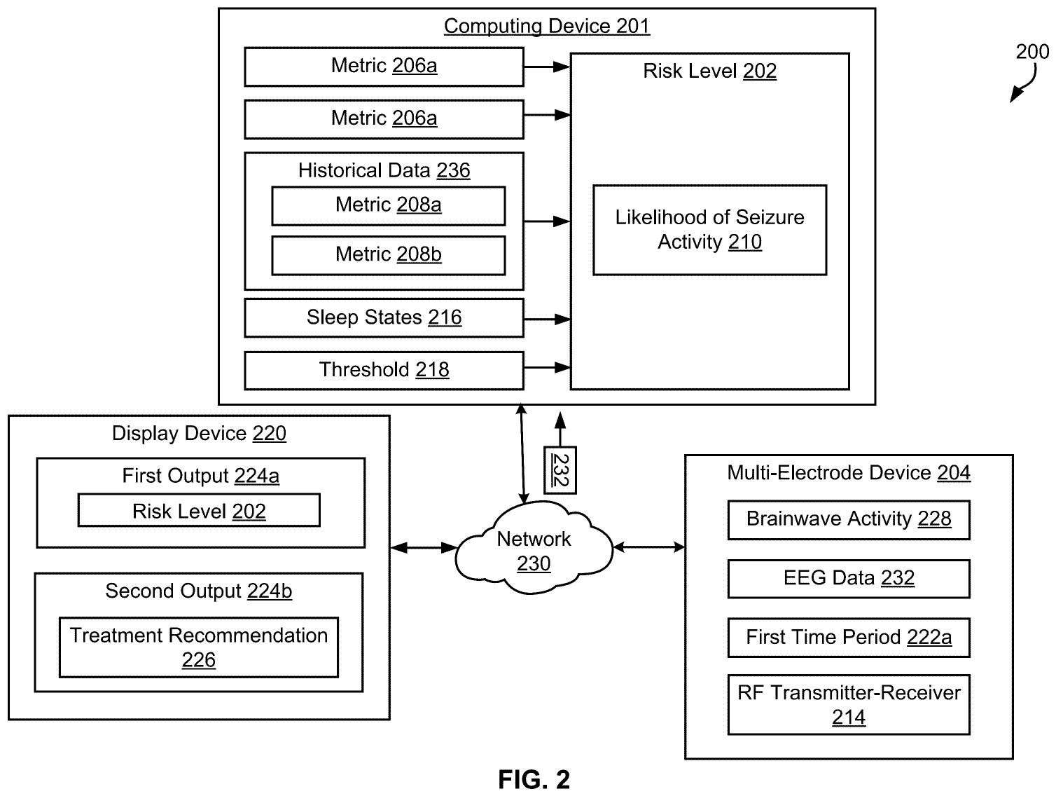

FIG. 2 is a block diagram of an example of a system 200 for identifying a risk level 202 for seizure activity based on sleep states 216 according to one example of the present disclosure. The risk level 202 can be a likelihood of a subject experiencing seizure activity (e.g., within a predefined time period). The system 200 can include a computing device 201, which can be communicatively coupled with a display device 220 and a multi-electrode device 204 for identifying the risk level 202 based on physiological data indicative of the sleep states 216 and providing the risk level 202 to a subject, physician, or another suitable user. The physiological data can include electroencephalogram (EEG) data 232, electromyogram (EMG) data, electrocardiogram (ECG) data, electrooculogram (EOG) data, or other suitable physiological data. The computing device 201 may communicate with the display device 220 and the multi-electrode device 204 via a network 230, such as a local area network (LAN) or the internet.

In some examples, the computing device 201 can receive the physiological data from the multi-electrode device 204 or other suitable devices or sensors. In an example, the multi-electrode device 204 can correspond to the multi-electrode device 104 of FIG. 1. Therefore, the computing device 201 may receive the physiological data, such as the EEG data 232, from an RF transmitter-receiver 114 associated with the multi-electrode device 204. The EEG data 232 can be indicative of brainwave activity 238 of the subject. The multi-electrode device 204 can also be associated with environmental sensors such as an accelerometer, an ECG, an EOG, an EMG, etc. for collecting additional physiological data. Additionally, the computing device 201 may receive the EEG data 232 for a first time period 222a, such as for one sleep cycle, for a series of sleep cycles (e.g., four subsequent sleep cycles), etc. The multi-electrode device 204 can, in addition to recording the EEG data 232, filter, amplify, transmit, or otherwise perform operations on the EEG data 232 or the additional physiological data to improve ingestion of the EEG data 232 or the additional physiological data at the computing device 201, thereby improving the efficiency and accuracy of the identification of the risk level 202.

Additionally, the computing device 201 may analyze the EEG data 232 or the additional physiological data to detect or otherwise differentiate between the sleep states 216. For example, the computing device 201 may collect, via the multi-electrode device 204, the EEG data 232 for a first time period 222a (i.e., an 8-hour period of sleep) and segment the EEG data 232 for the first time period 222a into epochs via, for example, a scanning window. The epochs may be analyzed by the computing device 201 to predict sleep states associated with each of the epochs. The epochs may initially be segmented into many, shorter time segments (e.g., 1 minute, 3 minutes, or 5-minute time segments) and then reorganized based on the predicted sleep states.

To analyze the epochs, the computing device 201 may further perform fast Fourier transform (FFT) or other suitable mathematical transformations, algorithms, or techniques to transform the EEG data 232 of each epoch to a frequency-domain. Once the EEG data 232 is transformed to the frequency-domain, additional features can be extracted such as peak amplitude, peak frequency, median frequency, etc. In some instances, the epochs may be further normalized or double normalized based on (for example) frequency information, amplitude information, power, intensity, or other suitable features of the EEG data. Moreover, the computing device 201 may perform short-time Fourier transform (STFT) or other suitable techniques to identify time-frequency-domain features from the EEG data 232, which can be dependent on both time-domain and frequency-domain characteristics. For example, the computing device 201 may perform the STFT to extract time-frequency-domain features such as average power, entropy, fractional energy, etc.

After analysis, the frequency information, amplitude information, time-frequency-domain features, etc. associated with each of the epochs can be correlated with sleep states to predict sleep states for each of the epochs. For example, the sleep states 216 can be characterized or defined as a wake sleep state, stage one, stage two, stage three, and rapid eye movement (REM) or the sleep states 216 can be characterized or defined as REM and non-REM. The computing device 201 can detect the sleep states 216 based the time-domain features, the frequency-domain features, the time-frequency-domain features, other suitable features, or a combination thereof as identified by the computing device 201 based on the EEG data 232. The features of the EEG data 232 can be indicative of characteristics of or changes in brainwave activity 228, which can correspond to the sleep states 216.

In some examples, detection algorithms may be configured by the computing device 201 in the time or frequency domain to detect signatures that support sleep state predictions. For example, the brainwave activity 228 for the stage three sleep state can include delta waves, which can exhibit low frequencies (i.e., a frequency band extending between one and four Hz) and high amplitudes (i.e., amplitudes extending between one hundred and two hundred μV) in the frequency-domain of the EEG data 232. Thus, if a first epoch includes the one to four Hz frequency band and amplitudes between one hundred and two hundred μV, a first sleep state for the first epoch can be predicted to be stage three.

In other examples, classification algorithms (i.e., K-Nearest neighbors, SVM, or other suitable classification algorithms) may be configured to predict the sleep states for each of the epochs by classifying the epochs based on the signatures that support sleep state predictions. The classification algorithms can be trained to predict the sleep states by inputting epochs labeled with sleep states or the classification algorithms can classify sleep states based on predefined rules. Therefore, for example, a classification algorithm may classify a second epoch with amplitudes in a three to eight Hz frequency band between fifty to one-hundred μV (i.e., Theta waves) as stage one sleep.

After the sleep states for each of the epochs are predicted, the computing device 201 may further determine metrics 206a-b associated with the sleep states 216. In some examples, the metrics 206a-b can be determined based on frequency patterns, amplitudes, or other suitable features extractable by the computing device 201 from the EEG data 232 and corresponding to the sleep states 216. For example, the metrics 206a-b may be a power, intensity, or other suitable attributes of a frequency band. The metrics 206a-b may be averaged, summed, or otherwise determined for one or more epochs associated with a particular sleep state. Additionally, in some examples, the metrics 206a-b can be statistical values obtained based on the predicted sleep states. For example, the metrics 206a-b can be a ratio of REM sleep to non-REM sleep, a percentage of time spent in a particular sleep state, etc. In a particular example, a first metric 206a can be a cumulative amount of time for which the subject was predicted to be the REM sleep state over the first time period 222a. The first metric 206a may be determined by adding time periods associated with each epoch for which it was predicted that the subject was in REM sleep. The first metric 206a can be an absolute amount of time (e.g., twenty minutes) or a relative amount of time (e.g., eight percent across the first time period 222a).

In another example, the first metric 206a can be an estimated amount of time in REM sleep for a sleep cycle or for a series of sleep cycles. Therefore, the epochs for the first time period 222a may be further grouped or organized by sleep cycle and, for each sleep cycle, a time period for which the subject was predicted to be in the REM sleep state can be determined. The first metric 206a may further be an average, median, mode, etc. of REM sleep across the series of sleep cycles.

Additionally, a second metric 206b can be an amount of time spent asleep (e.g., accumulation of stage one, stage two, stage three, and REM). In some examples, the metrics 206a-b may further include an amount of time spent in stage one, stage two, stage three, or a combination thereof. Additionally, the metrics 206a-b may include characteristics of sleep cycles (e.g., length of a sleep cycle or percentages for time spent in each sleep state of a sleep cycle) for the subject or other suitable metrics that can be calculated, estimated, or otherwise identified based on the EEG data 232 or other suitable physiological data.

The computing device 201 may further determine, based on the metrics 206a-b, the risk level 202. In some examples, the computing device 201 can implement artificial-intelligence techniques can be used to predict the risk level 202 based on the metrics 206a-b. The artificial-intelligence techniques may include using one or more models or rules to generate a subject-specific predictions based on the metrics 206a-b derived from one or more epochs for the first time period 222a. The rules can be defined to predict REM deprivation and/or seizure propensity based on the metrics 206a-b. For example, a rule can be defined to predict whether the subject is (or was) sleep deprived. For example, the rule may indicate that the subject is (or was) sleep deprived) if less than a threshold percentage (e.g., 10%, 15%, 20%, 25%, 30%, or 35%) of the epochs are predicted to be REM sleep. In another example, a rule may indicate that the subject was or is sleep deprived by identifying that a length of time for REM sleep as predicted by the detection algorithms for one or more epochs is less than a predefined threshold for healthy or normal sleep or for a learned threshold for the subject. The artificial-intelligence techniques may further make subject-specific predictions based on additional characteristics of the subject (i.e., age, previous diagnosis of a sleep or seizure associated disorder, prescribed medications, etc.).

In a particular example, the first metric 206a can be the relative amount of time for which the subject was predicted to be in REM sleep over the first time period 222a. Threshold amounts of time for REM sleep can be predefined based on age group. The threshold amounts of time for REM sleep may be a gradient such that each of the multiple thresholds amounts of REM sleep corresponds to a different level of risk. For example, for an age group of thirty to fifty years old, the threshold amounts of REM sleep can be relative times of twenty five percent, twenty percent, and fifteen percent. Thus, to illustrate risk level prediction, if a subject is thirty-five years old and a predicted, relative amount of time for REM sleep over the first time period 222a for the subject is fourteen percent, the computing device 201 may predict a high risk level for the subject.

Additionally, or alternatively, the computing device 201 may determine the risk level 202 based on historical data 236. For example, the historical data 236 can be previous EEG data associated with sleep for the subject (e.g., while the subject was on a different medication) or EEG data collected for a healthy population, a population of subjects with seizure history, a population of subjects diagnosed with epilepsy, or another suitable population for which physiological data can be obtained and metrics 208a-b can be determined. The metrics 208a-b can be the same metrics or otherwise reflective of the same features of the EEG data 232 as the metrics 206a-b. The metrics 208a-b may be labeled with seizure occurrence for a predefined time period or otherwise include an indication of seizure activity. Therefore, the metrics 208a-b, the historical data 236, or a combination thereof may be used for training the artificial intelligence techniques or for generating predefined rules for the artificial intelligence techniques.

In response to the determining the risk level 202, the computing device 201 can generate a first output 224a indicating the risk level 202. The first output 224a can be output for display at display device 220. The first output 224a can indicate the high-risk level, a moderate risk level, or a low risk level. In some examples, a likelihood associated with the risk level can also be displayed. For example, a low-risk level can be associated with a likelihood of less than thirty-three percent, a moderate-risk level can be associated with a likelihood of greater than thirty-three percent and less than sixty-six percent, and the high-risk level can be associated with a likelihood of greater than sixty-six percent. The likelihoods can be the likelihood of seizure activity 210 over a second time period 222b. For example, the second time period 222b can be an eight-hour time period that occurs subsequent to the first time period 222a. Additionally, in some examples, the first output 224a may provide the high-risk level in a first color, the moderate risk level in a second color, and the low risk level in a third color. In other examples, the first output 224a may include a percentage for the likelihood of seizure activity 210 or otherwise indicate the risk level 202 at the display device 220.

Additionally, or alternatively, the computing device 201 may generate a second output 224b, which can include a treatment recommendation 226. The computing device 201 may determine the treatment recommendation 226 based on the EEG data 232, additional physiological data, the risk level 202, electronic health records, other suitable data, or a combination thereof for the subject. The treatment recommendation 226 can be a medication, a dosage of medication, or another suitable treatment recommendation that can reduce the risk level 202 for the subject.

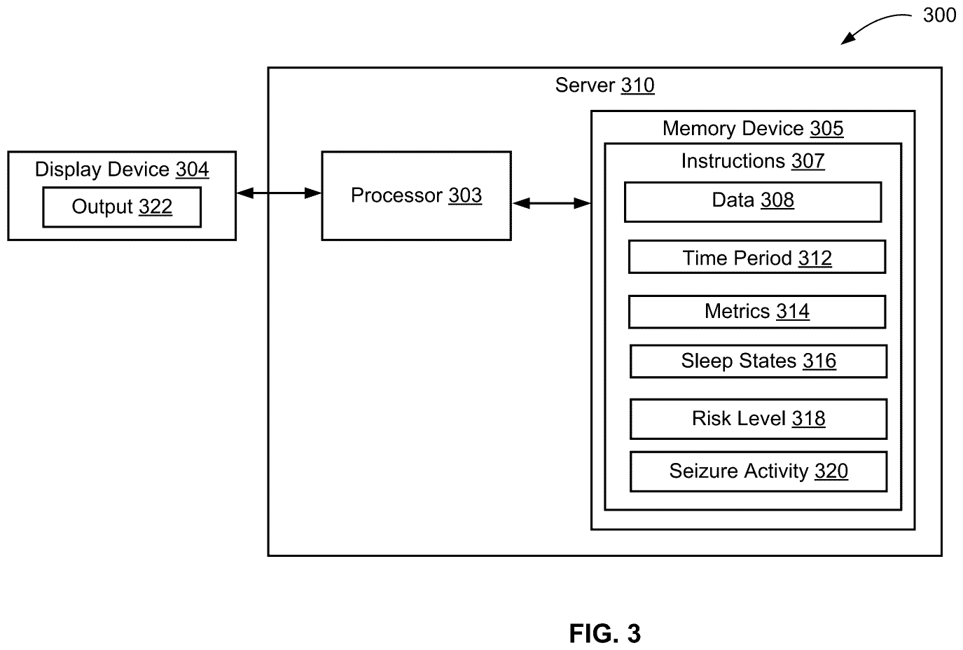

FIG. 3 is a block diagram of an example of a computing system 300 for identifying risk level for seizure activity based on sleep states according to one example of the present disclosure. The computing system 300 includes a processor 303 that is communicatively coupled to a memory device 305. In some examples, the processor 303 and the memory device 305 can be part of the same computing device, such as the server 310. In other examples, the processor 303 and the memory device 305 can be distributed from (e.g., remote to) one another.

The processor 303 can include one processor or multiple processors. Non-limiting examples of the processor 303 include a Field-Programmable Gate Array (FPGA), an application-specific integrated circuit (ASIC), or a microprocessor. The processor 303 can execute instructions 307 stored in the memory device 305 to perform operations. The instructions 307 may include processor-specific instructions generated by a compiler or an interpreter from code written in any suitable computer-programming language, such as C, C++, C#, Java, or Python.

The memory device 305 can include one memory or multiple memories. The memory device 305 can be volatile or non-volatile. Non-volatile memory includes any type of memory that retains stored information when powered off. Examples of the memory device 305 include electrically erasable and programmable read-only memory (EEPROM) or flash memory. At least some of the memory device 305 can include a non-transitory computer-readable medium from which the processor 303 can read instructions 307. A non-transitory computer-readable medium can include electronic, optical, magnetic, or other storage devices capable of providing the processor 303 with computer-readable instructions or other program code. Examples of a non-transitory computer-readable medium can include a magnetic disk, a memory chip, ROM, random-access memory (RAM), an ASIC, a configured processor, and optical storage.

The processor 303 can execute the instructions 307 to perform operations. For example, the processor 303 can receive data 308 indicative of brainwave activity over a time period 312. The processor 303 can also determine, based on the data 308, metrics 314 associated with sleep states 316 for a subject. Additionally, the processor 303 can determine, based on the metrics 314, a risk level 318 associated with seizure activity 320 for the subject. The processor 303 can further generate an output 322 for display at a display device 304. The output 322 can indicate the risk level 318.

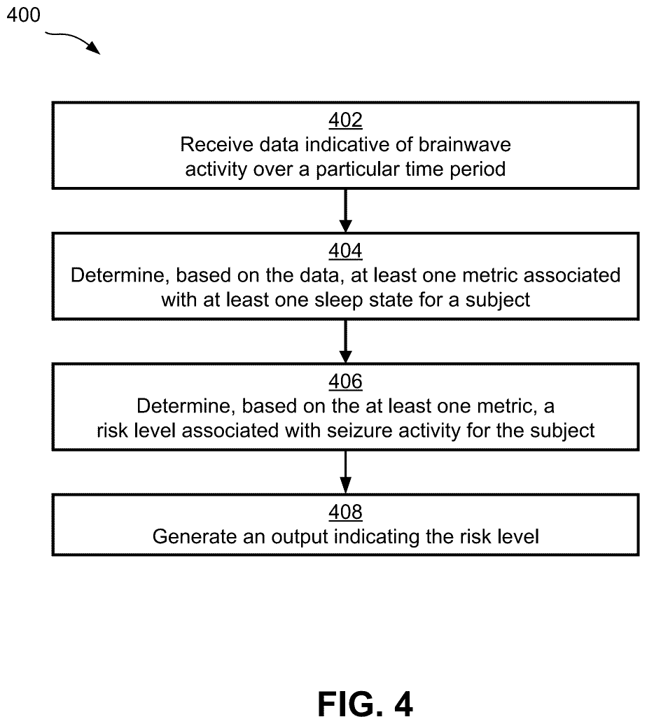

FIG. 4 is a flowchart of a process for identifying a risk level 202 for seizure activity based on sleep states 216 according to one example of the present disclosure. In some examples, a processor 303 can implement some or all of the steps shown in FIG. 4. Other examples can include more steps, fewer steps, different steps, or a different order of the steps than is shown in FIG. 4. The steps of FIG. 4 are discussed below with reference to the components discussed above in relation to FIGS. 2 and 3.

At block 402, the processor 303 can receive data indicative of brainwave activity 228 over a particular time period. The data can be electroencephalogram (EEG) data obtained via a multi-electrode device 204. The data can be received by the processor 303 from an RF transmitter-receiver associated with the multi-electrode device 204. In some examples, the particular time period can be a first time period 222a that is a predefined time period (e.g., an eight-hour time period) or can be defined by a number of sleep cycles (e.g., four sleep cycles). Additionally, the processor 303 may segment the data, in time, into epochs. The epochs can be any suitable length of time (e.g, 3 minutes, 5 minutes, 1 hour, etc.).

At block 404, the processor 303 can determine, based on the data, at least one metric 206a-b associated with at least one sleep state for the subject. The sleep states 216 can be characterized or defined by a wake sleep state, stage one, stage two, stage three, and rapid eye movement (REM) or the sleep states 216 can be characterized or defined by REM and non-REM. The sleep states 216 can be predicted via detection algorithms, classification algorithms, or other suitable algorithms executed by the processor 303 based on frequency patterns, amplitudes, or other suitable characteristics of the EEG data 232. In some examples, a sleep state can be predicted for each of the epochs for the first time period 222a. Then, the epochs may be reorganized based on the sleep states for which they are related.

After predicting the sleep states 216, the processor 203 may derive the metrics 206a-b from the EEG data 216 associated with the sleep states 216. For example, a first metric 206a can be an amount of time for a particular sleep state (e.g., REM). The amount of time for the particular sleep state can be cumulative over a predefined time period. In some examples, the amount of time for the particular sleep state can be an average amount of time for the particular sleep state per sleep cycle based on a series of sleep cycles. Additionally, the metrics 206a-b may include other suitable statistical values derived from the predicted sleep states or from the EEG data 232. For example, the metrics 206a-b can be a ratio of REM to non-REM sleep, an amount of time spent asleep (e.g., in stage one, stage two, stage three, and REM), an average frequency for brain waves during a particular sleep stage, an average amplitude for brain waves during the particular sleep stage, etc.

At block 406, the processor 303 can determine, based on the at least one metric 206a-b, a risk level 202 associated seizure activity for the subject. In some examples, the processor 303 can implement artificial intelligence techniques. to predict the risk level 202 based on the metrics 206a-b. The artificial-intelligence techniques may include using one or more models or rules to generate a subject-specific predictions based on the metrics 206a-b derived from one or more epochs for the first time period 222a. The rules can be defined to predict REM deprivation, other suitable abnormal sleep patterns, and/or seizure propensity based on the metrics 206a-b. For example, a rule can be defined to predict whether the subject is (or was) sleep deprived. For example, the rule may indicate that the subject is (or was) sleep deprived) if less than a threshold percentage (e.g., 10%, 15%, 20%, 25%, 30%, or 35%) of the epochs are predicted to be REM sleep. The rule may further define the thresholds based on age of the subject or other suitable characteristics. The rule may also include a gradient (i.e., multiple thresholds of varying levels) that can correspond to risk level. Thus, the rule can enable the processor to predict the risk level 202 based on first metric 206a.

In some examples, historical data 236 can be EEG data for a healthy population, for a population with a history of seizure activity, etc. or the historical data 236 can be EEG data obtained for the subject previously. Thus, the processor 303 may further use the historical data 236 for training the artificial intelligence techniques or for generating predefined rules, thresholds, etc. for determining the risk level 202.

At block 408, the processor 303 can generate an output 322 indicating the risk level 202. The output 322 can indicate a high-risk level, a moderate-risk level, or a low-risk level. In some examples, the low-risk level can be indicated by an output in a first color, the moderate-risk level can be indicated by an output in second color, and the high-risk level can be indicated by an output in a third color. The output 322 may also include the metrics 206a-b associated with the predicted sleep states, confidence values for the risk level, likelihood values (i.e., a percent chance of seizure activity), or other suitable indicators of the risk level 202. Additionally, in some examples, the processor 303 may generate a second output indicating the second time period. Additionally, or alternatively, the processor 303 may identify, based on the risk level 202, a treatment recommendation and can include the treatment recommendation in the output 322. The treatment recommendation can be used to reduce the risk level 202 for the subject.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

What is claimed is:

1. A computer-implemented method comprising:
receiving data indicative of brainwave activity over a particular time period;
determining, based on the data, at least one metric associated with at least one sleep state for a subject;
determining, based on the at least one metric, a risk level associated with seizure activity for the subject; and
generating an output indicating the risk level.

2. The computer-implemented method of claim 1, wherein the at least one metric is an amount of time for a particular sleep state.

3. The computer-implemented method of claim 2, wherein the particular sleep state is a rapid eye movement (REM) state of sleep.

4. The computer-implemented method of claim 1, wherein the data is received from a RF transmitter-receiver associated with a multi-electrode device.

5. The computer-implemented method of claim 1, wherein the particular time period is a first time period, and wherein the risk level is a prediction of a likelihood of the subject experiencing the seizure activity for a second time period, wherein the second time period occurs subsequent to the first time period.

6. The computer-implemented method of claim 1, wherein the output is a first output and further comprising:
identifying, based on the risk level, a treatment recommendation; and
generating a second output indicating the treatment recommendation, the treatment recommendation usable to reduce the risk level.

7. The computer-implemented method of claim 1, wherein the at least one metric is a first metric and determining, based on the at least one metric, the risk level further comprises:
determining a second metric for a healthy population based on historical data;
identifying a statistically significant difference between the first metric and the second metric based on a comparison of the first metric and the second metric; and
determining, based on the statistically significant difference, the risk level.

8. The computer-implemented method of claim 1, wherein generating the output indicating the risk level further comprises:
providing the output indicating a high-risk level in a first color;
providing the output indicating a moderate risk level in a second color; and
providing the output indicating a low risk level in a third color.

9. A system comprising:
one or more data processors; and
a non-transitory computer readable storage medium containing instructions which, when executed on the one or more data processors, cause the one or more data processors to:
receive data indicative of brainwave activity over a particular time period;
determine, based on the data, at least one metric associated with at least one sleep state for a subject;
determine, based on the at least one metric, a risk level associated with seizure activity for the subject; and
generate an output indicating the risk level.

10. The system of claim 9, wherein the at least one metric is an amount of time for a particular sleep state.

11. The system of claim 10, wherein the particular sleep state is a rapid eye movement (REM) state of sleep.

12. The system of claim 9, wherein the data is received from a RF transmitter-receiver associated with a multi-electrode device.

13. The system of claim 9, wherein the particular time period is a first time period, and wherein the risk level is a prediction of a likelihood of the subject experiencing the seizure activity for a second time period, wherein the second time period occurs subsequent to the first time period.

14. The system of claim 9, wherein the output is a first output and further comprising:
identifying, based on the risk level, a treatment recommendation; and
generating a second output indicating the treatment recommendation, the treatment recommendation usable to reduce the risk level.

15. The system of claim 9, wherein the at least one metric is a first metric and determining, based on the at least one metric, the risk level further comprises:
determining a second metric for a healthy population based on historical data;
identifying a statistically significant difference between the first metric and the second metric based on a comparison of the first metric and the second metric; and
determining, based on the statistically significant difference, the risk level.

16. The system of claim 9, wherein generating the output indicating the risk level further comprises:
providing the output indicating a high-risk level in a first color;
providing the output indicating a moderate risk level in a second color; and
providing the output indicating a low risk level in a third color.

17. A computer-program product tangibly embodied in a non-transitory machine-readable storage medium, including instructions configured to cause one or more data processors to:
receive data indicative of brainwave activity over a particular time period;
determine, based on the data, at least one metric associated with at least one sleep state for a subject;
determine, based on the at least one metric, a risk level associated with seizure activity for the subject; and
generate an output indicating the risk level.

18. The computer-program of claim 17, wherein the at least one metric is an amount of time for a particular sleep state.

19. The computer-program of claim 18, wherein the particular sleep state is a rapid eye movement (REM) state of sleep.

20. The computer-program of claim 17, wherein the data is received from a RF transmitter-receiver associated with a multi-electrode device.

21. The computer-program of claim 17, wherein the particular time period is a first time period, and wherein the risk level is a prediction of a likelihood of the subject experiencing the seizure activity for a second time period, wherein the second time period occurs subsequent to the first time period.

22. The computer-program of claim 17, wherein the output is a first output and further comprising:
identifying, based on the risk level, a treatment recommendation; and
generating a second output indicating the treatment recommendation, the treatment recommendation usable to reduce the risk level.

23. The computer-program of claim 17, wherein the at least one metric is a first metric and determining, based on the at least one metric, the risk level further comprises:
determining a second metric for a healthy population based on historical data;
identifying a statistically significant difference between the first metric and the second metric based on a comparison of the first metric and the second metric; and
determining, based on the statistically significant difference, the risk level.

24. The computer-program of claim 17, wherein generating the output indicating the risk level further comprises:
providing the output indicating a high-risk level in a first color;
providing the output indicating a moderate risk level in a second color; and
providing the output indicating a low risk level in a third color.
